(19) United States
(12) Patent Application Publication
(10) Pub. No.: US 2016/0242643 A1
(43) Pub. Date: Aug. 25, 2016

(54) Transparent Camera for Imaging the Eye

(71) Applicant: Gregory Allan Sprehn, Oceanside, CA (US)

(72) Inventor: Gregory Allan Sprehn, Carlsbad, CA (US)

(73) Assignee: RIVENDELL HEIGHTS, INC., Oceanside, CA (US)

(21) Appl. No.: 14/626,869

(22) Filed: Feb. 19, 2015

(51) Int. Cl. A61B 3/14; A61B 3/12; A61B 3/00; A61B 3/113

(52) U.S. Cl. CPC A61B 3/14; A61B 3/113; A61B 3/12; A61B 3/0025; A61B 3/0075; A61B 3/0083; A61B 3/005; G02B 27/14

(57) ABSTRACT

A method of illuminating and imaging the eye on or near the visual axis, without interrupting vision, useful for gaze tracking, micro-tracking, choroid self-examination, and long term fixation

### BACKGROUND OF THE INVENTION

1. Gaze-tracking algorithms often require an image of the front of the eye from canthus to canthus in order to locate and analyze the pupil shape and position. Cameras to provide the tracking images are located about 15 degrees off axis, out of the central view. However, this offset reduces accuracy and limits the effective tracking area. An image taken directly in front of the eye is ideal for tracking, but blocks the view.
2. Gaze-tracking with the pupil image is limited to about one degree accuracy, even with glint tracking. Tracking gaze by viewing the position of the foveal choroid is much more accurate, but challenging to accomplish. Many cameras designed to look inside of the eye can provide an image of the choroid using infrared light, but they also block the view.
3. Modern cameras that look into the eye display the image on a computer monitor. Viewing the image of the inside of one's own eye is prevented by the opacity of the camera taking the image. The ability to easily image and view the inside of one's own eye without assistance would enable low-cost self-screening for eye-health.
4. Fixation during long procedures such as mfERG and dark adaptation is difficult to maintain. Small, fixed light sources seem to vanish with time. Fixating on a live image of one's own optic disc provides a captivating target with targeting self-correction. Observing natural micro-saccades is relaxing, providing long term, stress-free fixation.

An invisible camera is needed to image the front and inside of the eye, without blocking the view.

### SUMMARY OF THE INVENTION

The object of the present invention is to provide a line-of-sight, real time image of the front and inside of the eye, which does not interfere with viewer observation.

It is a particular objective of the present invention to use light waves longer than 800 nanometers to illuminate the eye.

It is a particular objective of the present invention to use a camera that is sensitive to light waves longer than 800 nanometers.

It is a particular objective of the present invention to use 2 optical elements in the visual path, one to inject light into the optical path to illuminate the eye, and the other to extract enough reflected light to form an image of the eye from temporal to nasal canthus.

Another feature of the present invention is to use a wavelength-selective mirror as the optic in the illumination path.

Another feature of the present invention is to use a partially reflective beamsplitter as the optic in the image path.

Another feature of the present invention is to limit corneal reflex of infrared illumination light to appear as one glint, to simplify gaze-tracking algorithms that require the glint.

Another feature of the present invention is to apply an anti-reflective coating to the optics to maximize transmission at the desired wavelengths and minimize unwanted reflections.

Another feature of the present invention is to use an electronically tunable lens to focus the image, instead of mechanical focusing methods that reposition optical elements.

Another feature of the present invention is to provide aperture adjustment to improve image quality with bright images.

Another feature of the present invention is to properly locate beam dumps and non-reflective finishes to absorb unwanted light.

Another feature of the present invention is to illuminate the retina with visible wavelengths of light using a wavelength-selective beamsplitter in the imaging path. Sources of visible light include, but are not limited to LED's for ERG or VEP stimulation or bleaching and lasers for therapy or photocoagulation.

Another feature of the present invention is to vary the duration and intensity of the source of the visible wavelengths of light.

Another feature of the present invention is to provide an image of the choroid.

Another feature of the present invention is to allow the viewer to view a wall-mounted computer display.

Another feature of the present invention is to vary the apparent size of a wall-mounted display with turret-mounted lenses.

Another feature of the present invention is to provide eye tracking while the viewer is stimulated by images on the display.

Another feature of the present invention is to reflect bright light from the surface of the display to provide bleaching of selective areas of the viewer's photoreceptors.

Another feature of the present invention is to provide a microdisplay in the viewing path.

Another feature of the present invention is to vary the apparent size of the microdisplay in the viewing path.

Another feature of the present invention is to provide eye tracking while the viewer is stimulated by images on the microdisplay.

Another feature of the present invention is to reflect bright light from the surface of the microdisplay to provide bleaching of selective areas of the viewer's photoreceptors.

Another feature of the present invention is to display the viewer's own choroid image on the display or microdisplay.

Another feature of the present invention is to present the choroid image off axis up to 15 degrees.

Another feature of the present invention is to position the choroid camera off axis up to 15 degrees.

Another feature of the present invention is to apply filters in the display path to attenuate the visible light, or polarize or depolarize, the visible light reaching the viewer.

Another feature of the present invention is to provide corrective optics at the viewport.

### BRIEF DESCRIPTION OF THE DRAWINGS

FIG. 1 shows a side view of a configuration with the front-of-the-eye camera and the microdisplay

FIG. 2 shows a side view of a configuration with the front-of-the-eye camera and a wall display

FIG. 3 shows a side view of anatomical landmarks on the eye

FIG. 4 shows two transparent optic elements positioned along the viewing axis

FIG. 5 shows the infrared light source path

FIG. 6 shows the glint path from the cornea

FIG. 7 shows the front-of-the-eye camera imaging path

FIG. 8 shows a representative image at the front-of-the-eye camera focal plane

FIG. 9 shows the video adapter block diagram

FIG. 10 shows the cold mirror visible light (bleach, laser) path

FIG. 11 shows a side view of a configuration similar to FIG. 1, with the front-of-the-eye camera relocated to add a choroid camera

FIG. 12 shows the choroid camera imaging path

FIG. 13 shows a representative image at the choroid camera focal plane for the configuration of FIG. 12

FIG. 14 shows the viewer looking 7.5 degrees to the side

FIG. 15 shows a representative image at the choroid camera focal plane for the configuration of FIG. 14

FIG. 16 shows a top view of the camera assembly

FIG. 17A shows a top view of the configuration with the display aligned with the viewing axis and the camera assembly aligned 15 degrees from the viewing axis as applied to the left eye. FIG. 17B is as applied to the right eye

FIG. 18 shows a representative image at the choroid camera focal plane for the configuration of FIG. 17B.

FIG. 19 shows the turret lens configuration to vary magnification of a wall-mounted display

FIG. 20 shows the viewing path to the microdisplay

FIG. 21A and FIG. 21B show variation of the magnification of the microdisplay with position of the optical components

### DESCRIPTION OF THE INVENTION

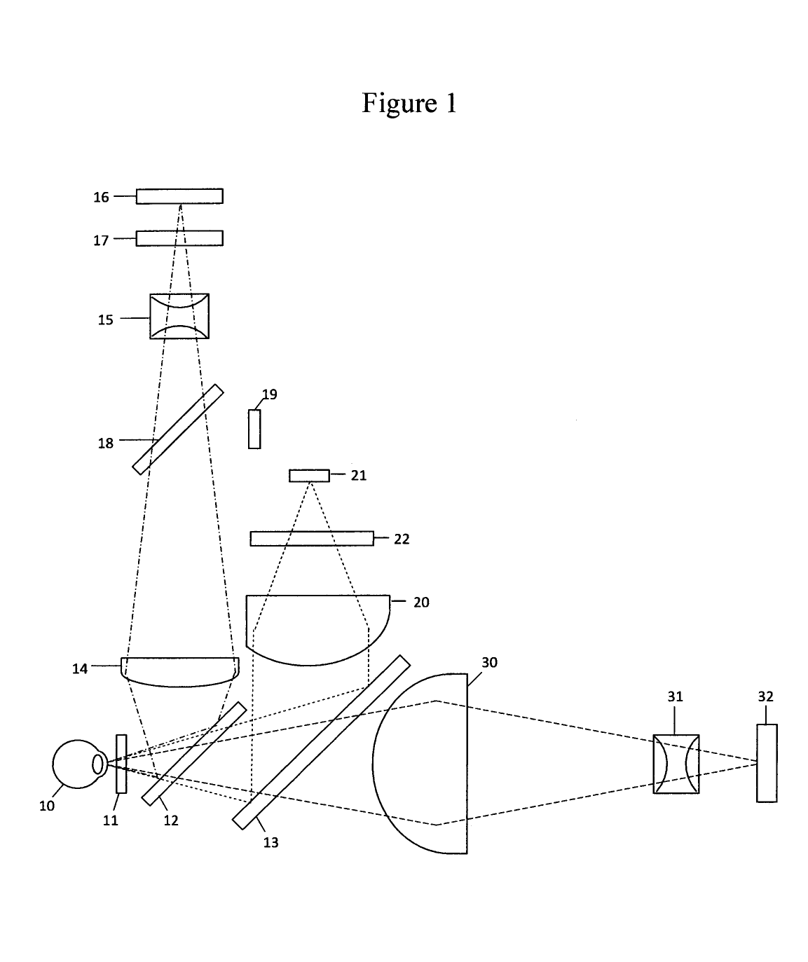

Refer to FIG. 1 for an overview. As a starting point and as part of this innovation, a light source 21 that provides infrared light from a tungsten-halogen filament supplied by regulated AC or DC current, or light emitting diodes, or a gaseous medium arc lamp, fitted with wavelength-selective filters 22 to remove visible and/or ultraviolet photons from the light spectrum, is introduced into an imaging path with a wavelength-selective mirror 13 through lens 20. The light source is sized and driven to deliver the optimum intensity of light required by the application.

In a preferred embodiment, the infrared light source uses a D-shaped lens 20 in the illumination. The desired viewing area of the choroid is primarily nasal, to view the optic disc, and thus does not need to be radially symmetric. A D-shaped lens allows the illumination and imaging paths to be closer together than they would be if a circular lens of the same diameter was used. Placing the illumination path closer to the imaging path reduces the size requirement on optic 13, as the required dimensions to maintain a specified field of view increase with distance.

The viewer's eye 10 is located on the horizontal optical axis before viewing port 11. The viewer can see through both optic 12 and 13. In FIG. 1, the viewer sees a microdisplay 32, viewed through relay 31 and projection lens 30.

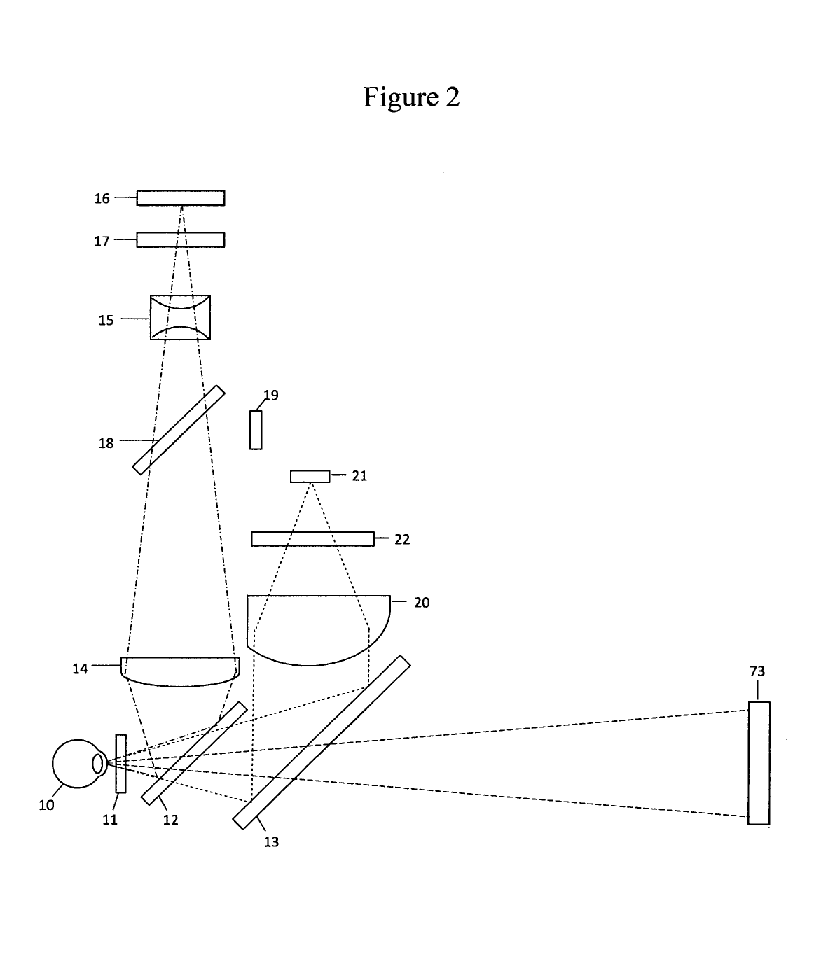

In FIG. 2, the viewer sees a larger wall display 73 located further away, without 30, 31 and 32.

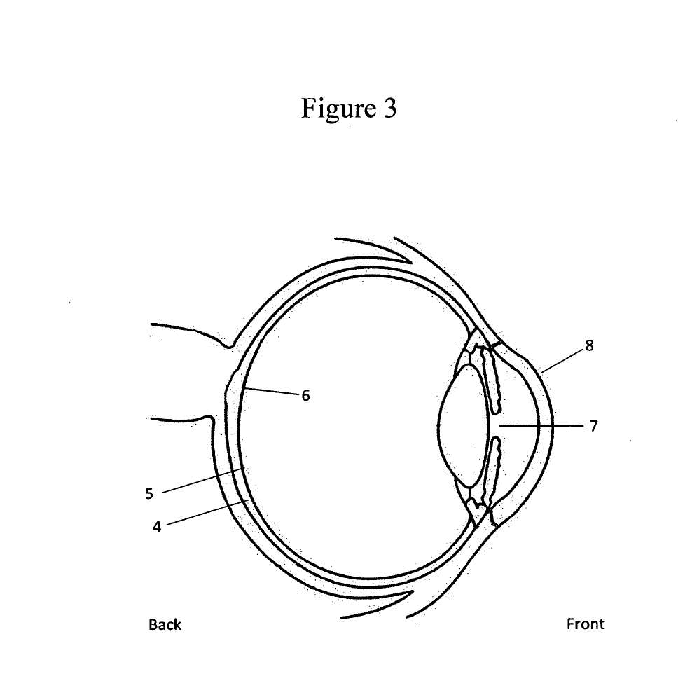

The Viewer's eye 10 is represented by FIG. 3, illustrating the choroid 4, retina 5, optic disc 6 at the back of the eye. The front of the eye shows pupil 7 and cornea 8.

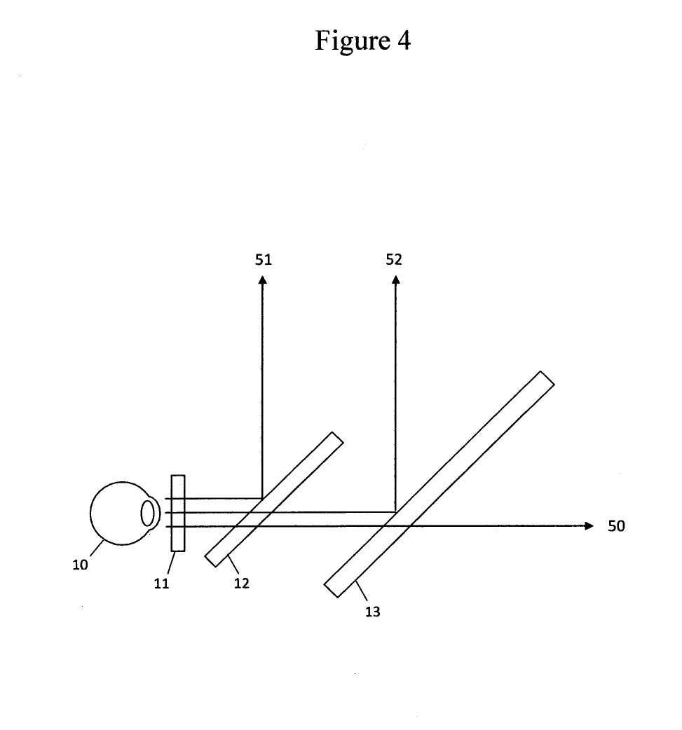

The imaging optical path 51 and the illumination optical path 52 are parallel to each other, and perpendicular to the viewing axis 50. Paths 51 and 52 are co-aligned with the viewing axis 50 at the viewing port 11 and at the viewer's eye 10. Refer to FIG. 4.

In a preferred embodiment, optic 12 is a partially reflective 45-degree beamsplitter coated to minimize backside reflections in the near infrared.

In a preferred embodiment, optic 13 is a wavelength-selective reflector, also known as a hot mirror, transparent to visible yet reflective of infrared light.

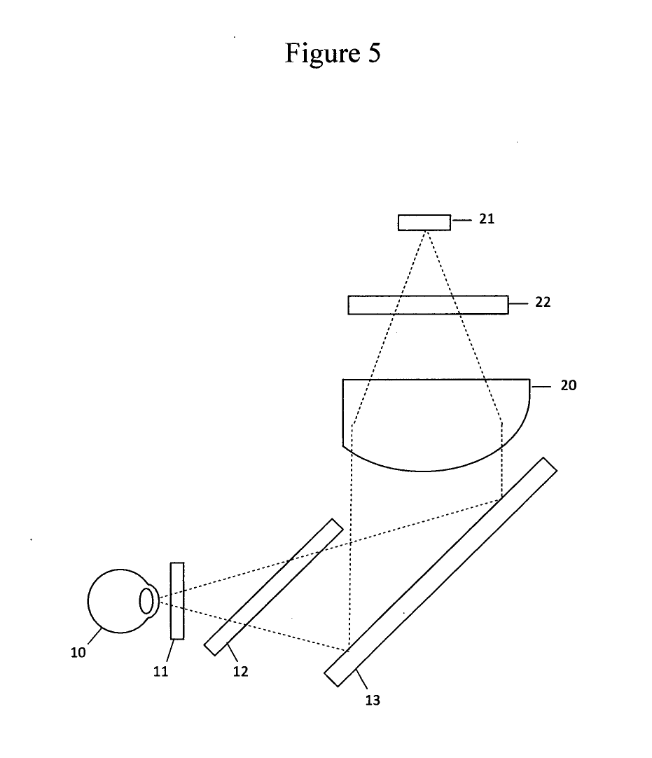

In a preferred embodiment, a single 5-watt LED is used for the infrared light source 21. Refer to FIG. 5.

Infrared light emitted by 21 and converged by 20 reflects from 13, passes through 12 and 11 and illuminates the viewer's eye 10.

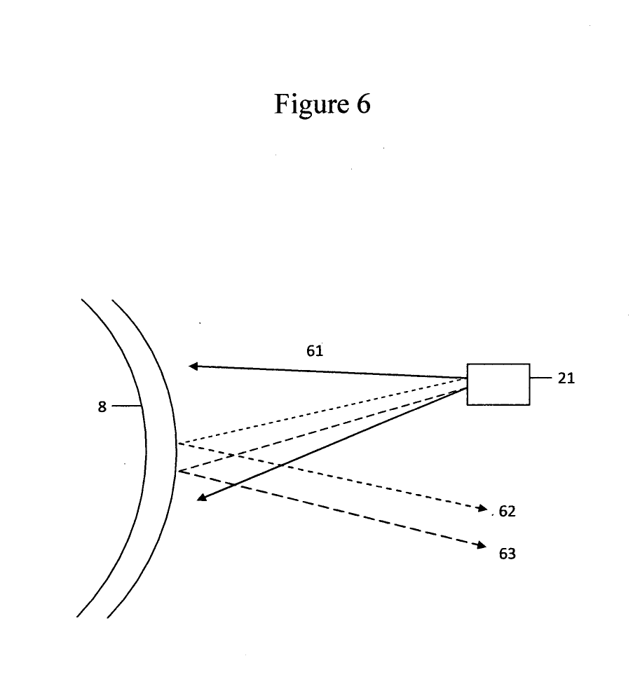

Infrared radiation, longer than 800 nm, is emitted by 21 and illuminates the cornea 8 with a cone of light 61. Refer to FIG. 6. The extent of 21 must be limited such that the extreme glints 62 and 63 appear as a single source.

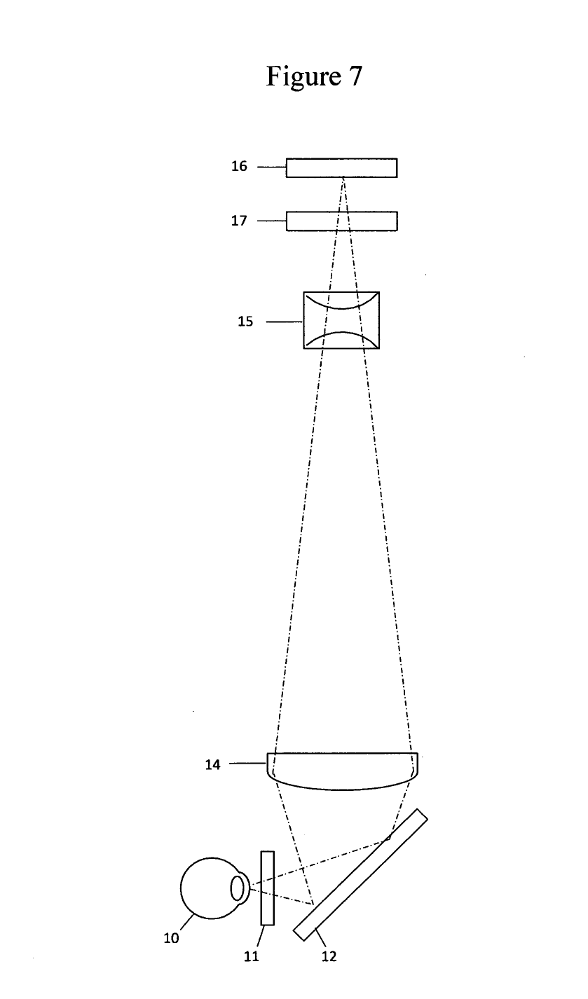

Infrared light reflected from the front of the eye 10 passes back through 11 and reflects from 12, passing through field lens 14 and relay 15 and then through filter 17 to camera 16. Refer to FIG. 7.

In a preferred embodiment, a wavelength-selective filter 17, transparent to infrared and opaque to visible light, is located before the camera.

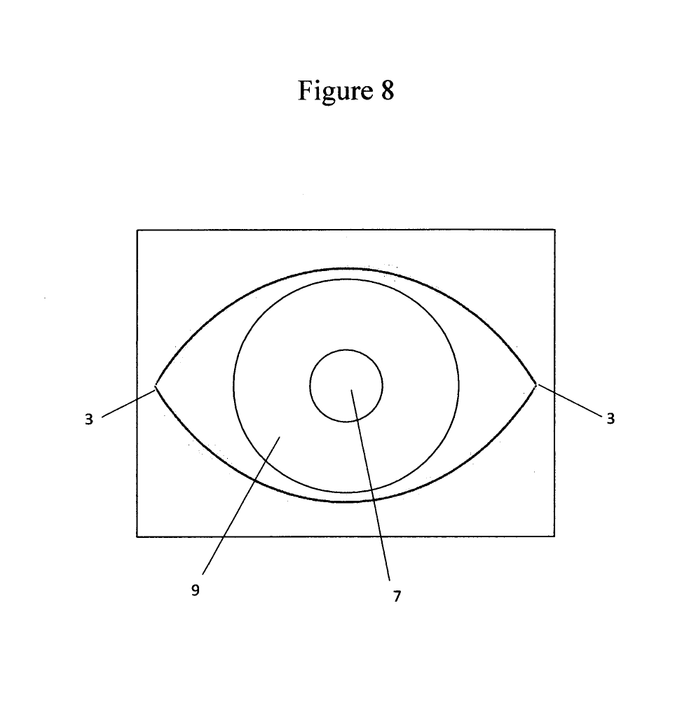

The image of the front of the eye at the focal plane of camera 16 is represented schematically in FIG. 8, illustrating canthus 3 at either side, pupil 7, and iris 9.

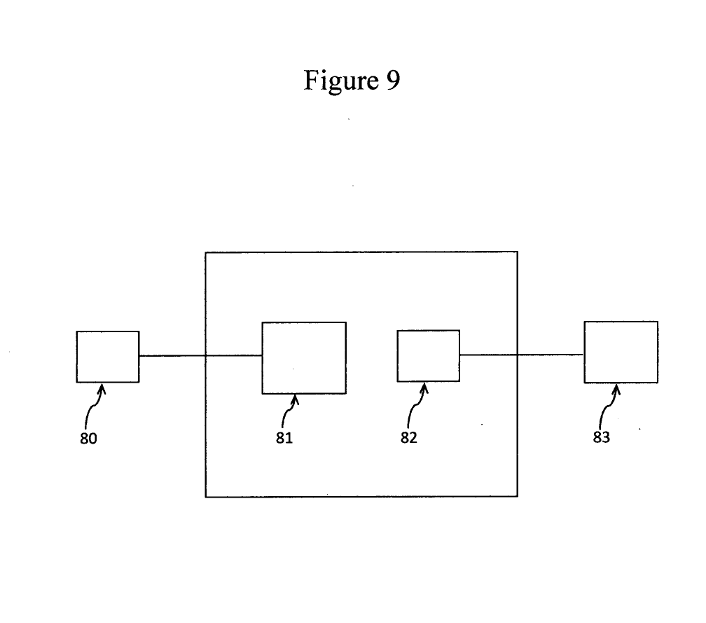

Also and a part of this invention, to accommodate specific, oversize, inconvenient eye-tracking camera requirements, a video adapter as shown in FIG. 9 is provided. The canthus to canthus eye image is obtained with a camera 80 suited to mounting inside of the imaging part of this invention, reproduced on a display 81 inside of an otherwise dark box, and then viewed through the air by the eye-tracking camera 82 connected to the eye-tracking computer 83.

In a preferred embodiment, lenses can be positioned between the eye-tracking camera and the display to change the apparent size of the image or correct other imaging abnormalities.

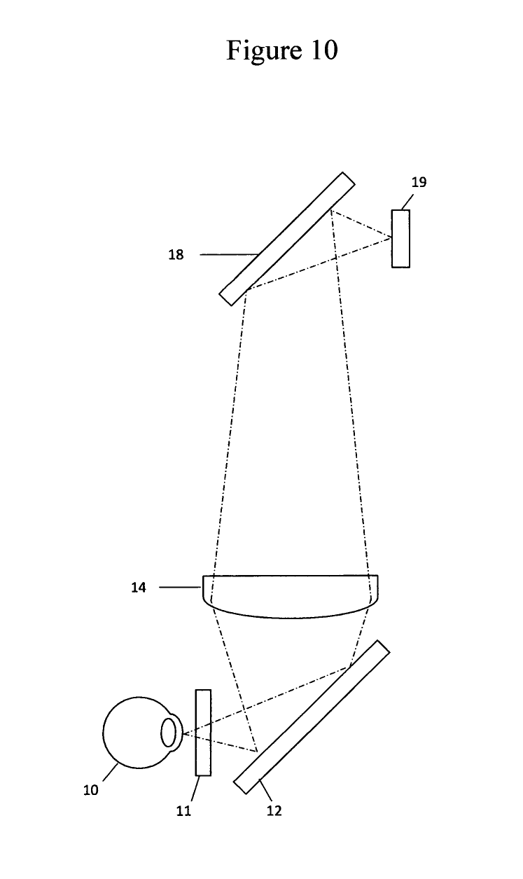

Also and a part of this invention, a wavelength-selective beamsplitter 18, also known as a cold-mirror, transparent to infrared light and reflecting visible light, is placed in the imaging path before the camera to introduce light from a visible source 19 towards the viewer. Refer to FIG. 10.

In a preferred embodiment, duration, intensity, and areal size on the retina of the visible light are well-controlled to produce a localized bleaching of viewer photoreceptors.

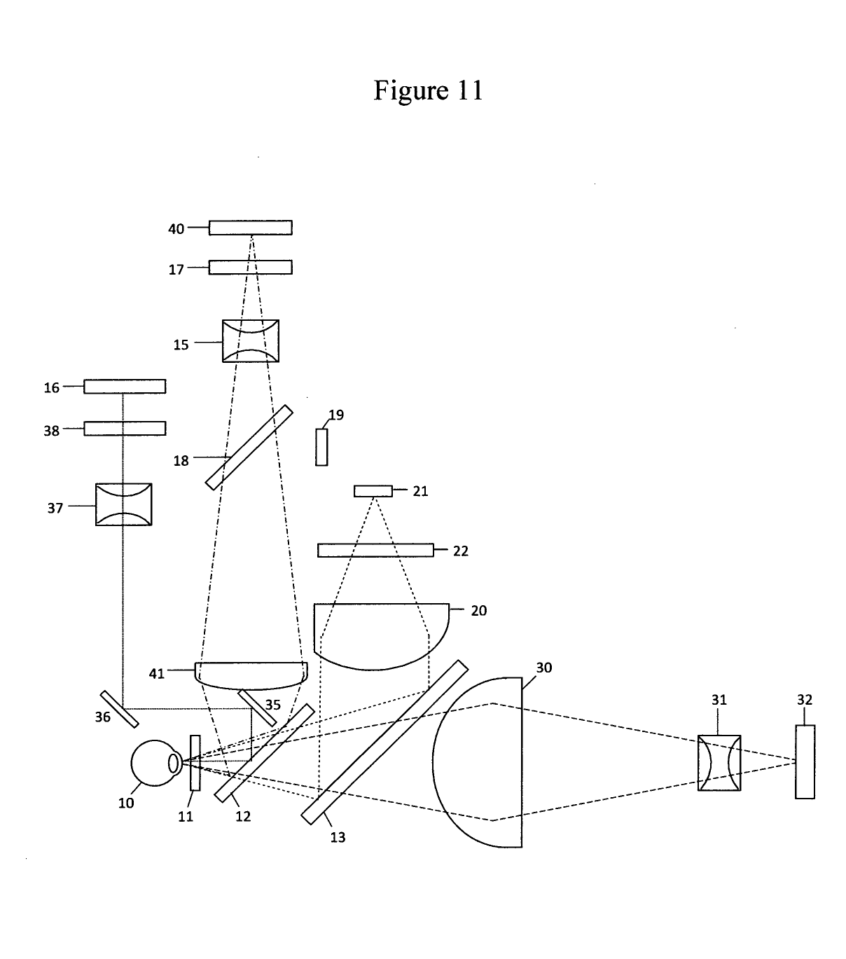

Also and a part of this invention is the addition of a choroid camera 40. Refer to FIG. 11.

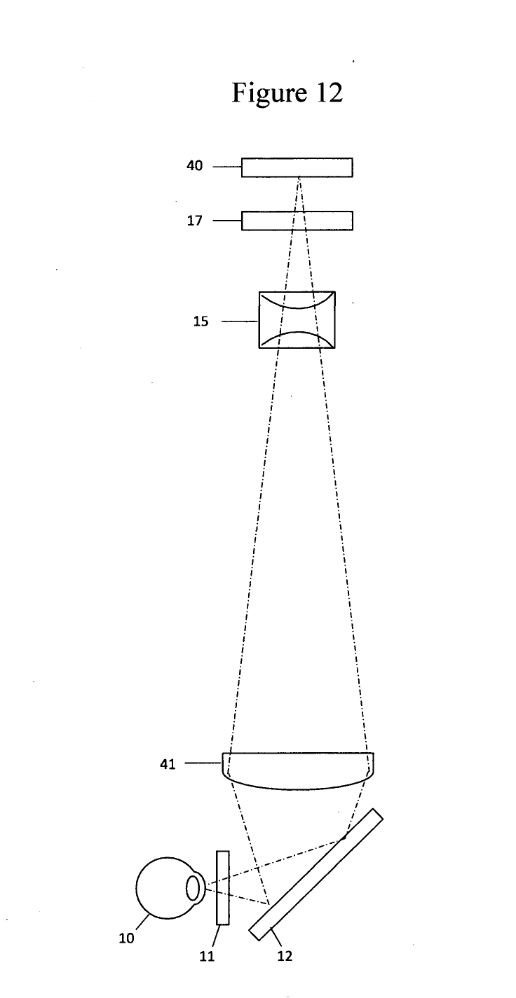

Infrared light reflected from the foveal choroid 4 passes out of the eye 10 and back through 11 to reflect from 12, passing then through field lens 41 and relay 15 and then through filter 17 to camera 40. Refer to FIG. 12.

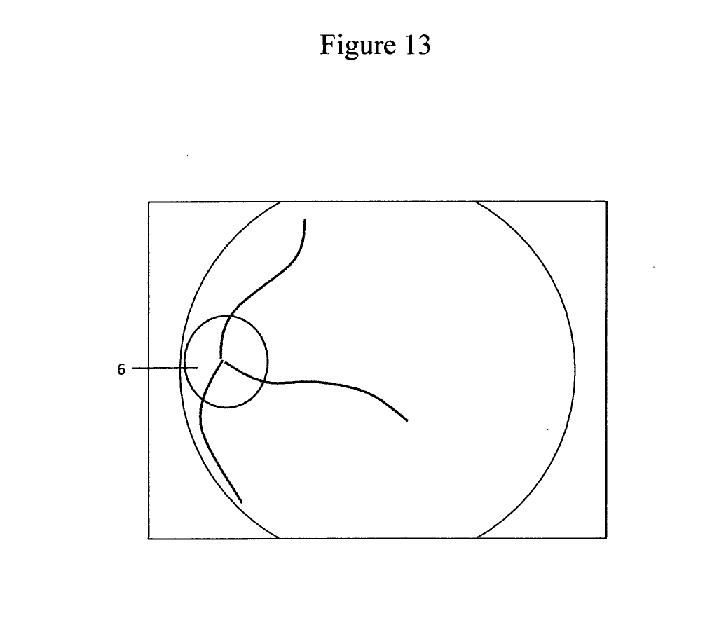

Anatomically, the optic disc is located 15 degrees from central view. FIG. 13 shows a representative image of the eye with the viewer looking straight ahead and the disc 15 degrees from the viewing axis.

In a preferred embodiment, a polarizer is positioned at optic 22 to polarize the illumination and a cross polarizer is positioned near 17 in the choroid camera 40 path to reduce the corneal reflex from the infrared light entering the eye.

A proper image of the front of the eye cannot be formed with the choroid field lens 41 in place. The front-of-the-eye image is extracted with an infrared beamsplitter 35 and fold mirror 36 positioned between optic 12 and lens 41, to obtain the required view. The image of the front of the eye at the focal plane for camera 16 is formed through relay 37 and optic 38. Refer again to FIG. 11.

In a preferred embodiment, the beamsplitter 35 is a microscope cover glass with about 4% reflectivity.

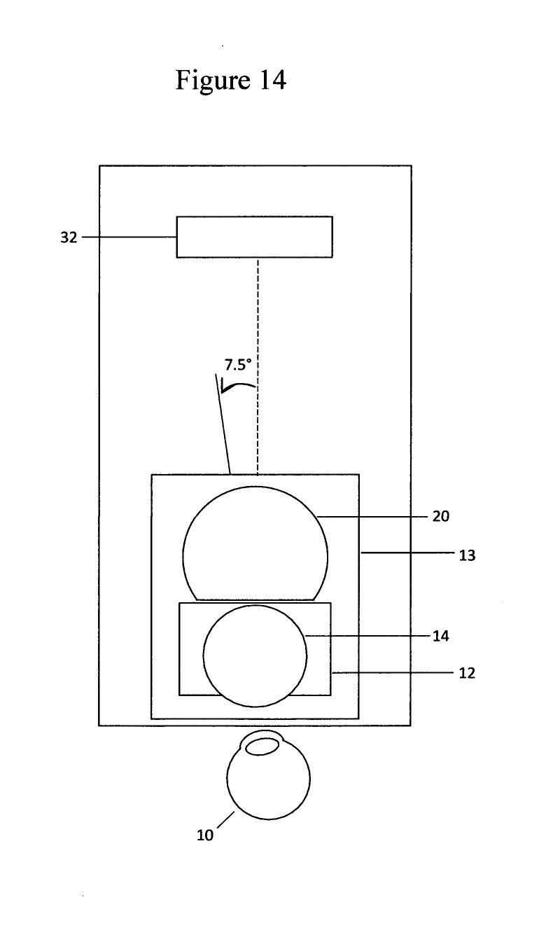

FIG. 14 shows one method of optic disc self-examination of the right eye. The viewer's gaze is purposely directed 7.5 degrees to the side to align the view with the image of the disc.

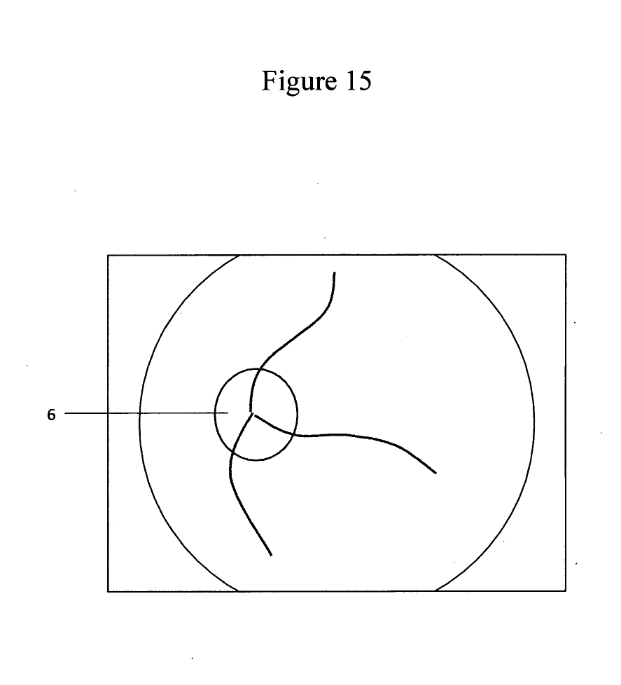

FIG. 15 is a representative image at the choroid camera focal plane for the configuration of FIG. 14. The disc is shown 7.5 degrees off center.

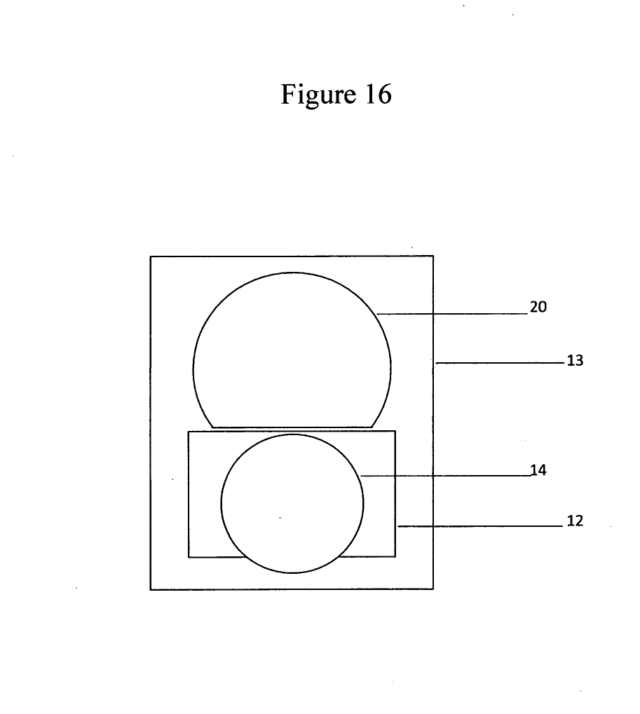

Also and a part of this invention is the ability to change the relative horizontal angle of the camera assembly with respect to central view. FIG. 16 shows the camera assembly alone.

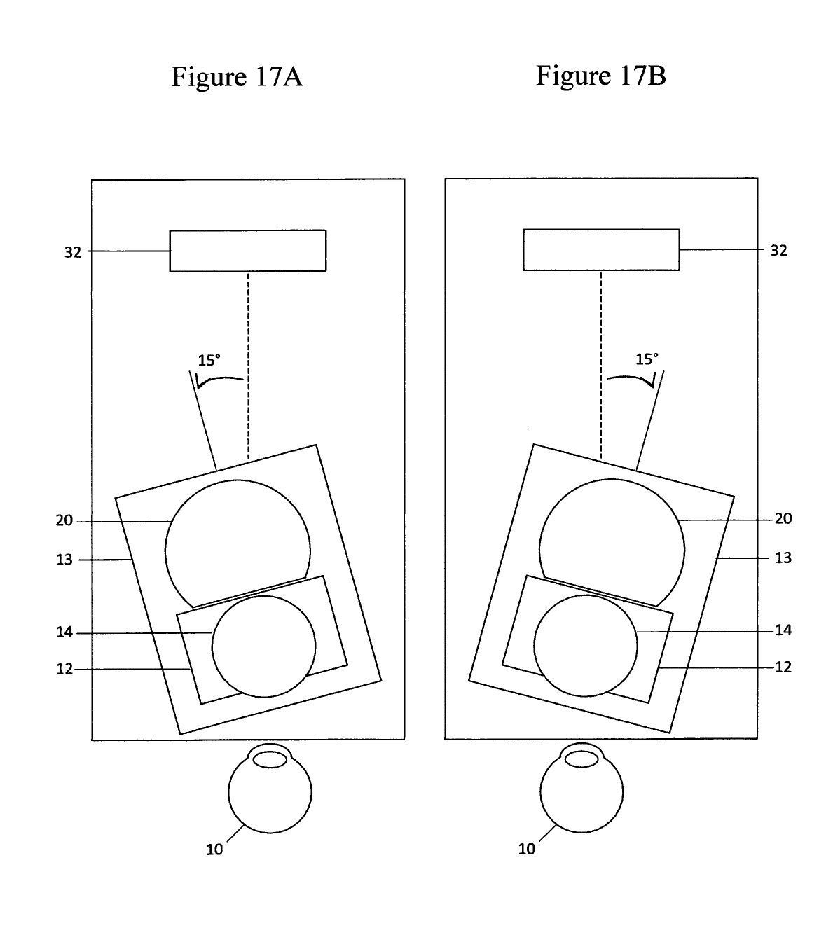

In a preferred embodiment, the entire vertical camera assembly can be rotated through an arc centered on the vertical axis that passes through the entrance point to the eye, for the purpose of imaging the eye from 15 degrees to the side of the viewer's central viewing axis 50. Refer to FIG. 17A for the left eye and FIG. 17B for the right eye. The viewer is then able to comfortably observe a live image of their own disc directly in front of them.

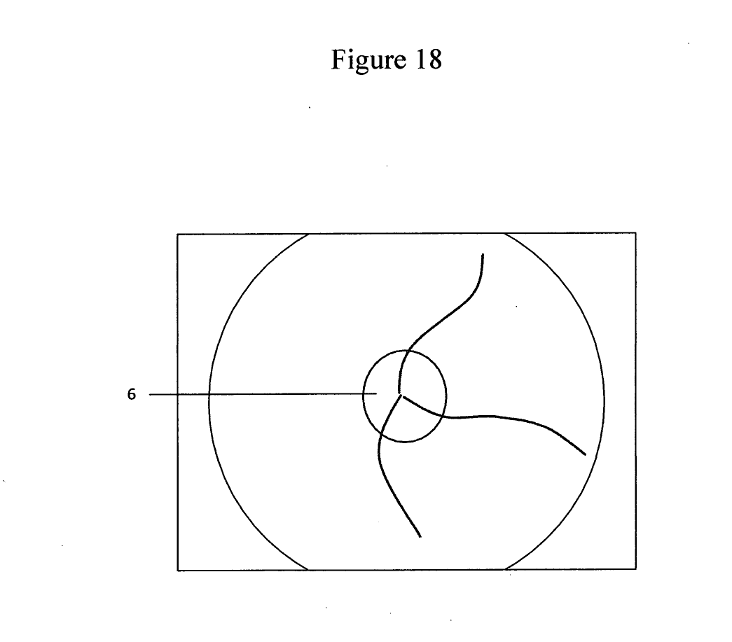

FIG. 18 is a representative image at the choroid camera focal plane for the configuration of FIG. 17A. The optic disc is centered.

In a preferred embodiment, when viewing a wall display 73, lenses can be introduced between optic 13 and the display 73 to change the apparent size to the viewer.

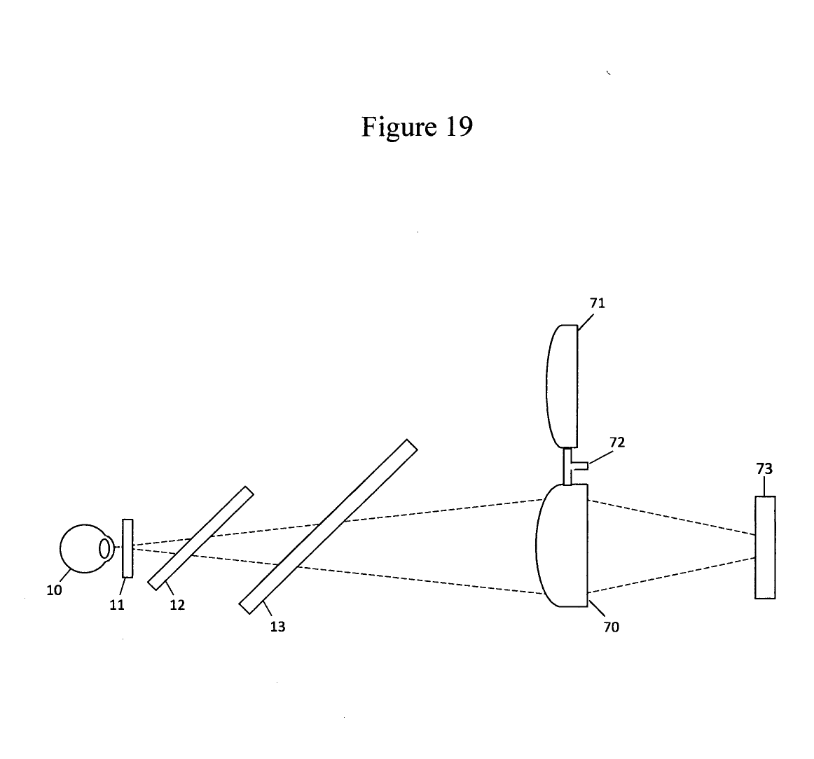

In a preferred embodiment, one or more lenses 70 and 71 are mounted to a turret 72 and rotated into view as needed to change the apparent size of the display 73 to the viewer. Refer to FIG. 19.

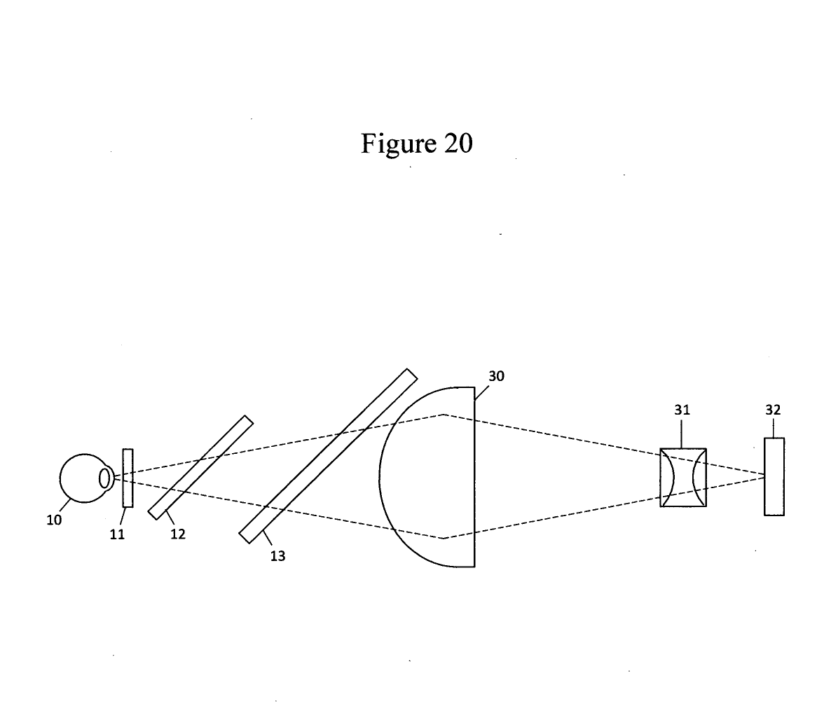

The viewing path to the microdisplay configuration is shown in FIG. 20.

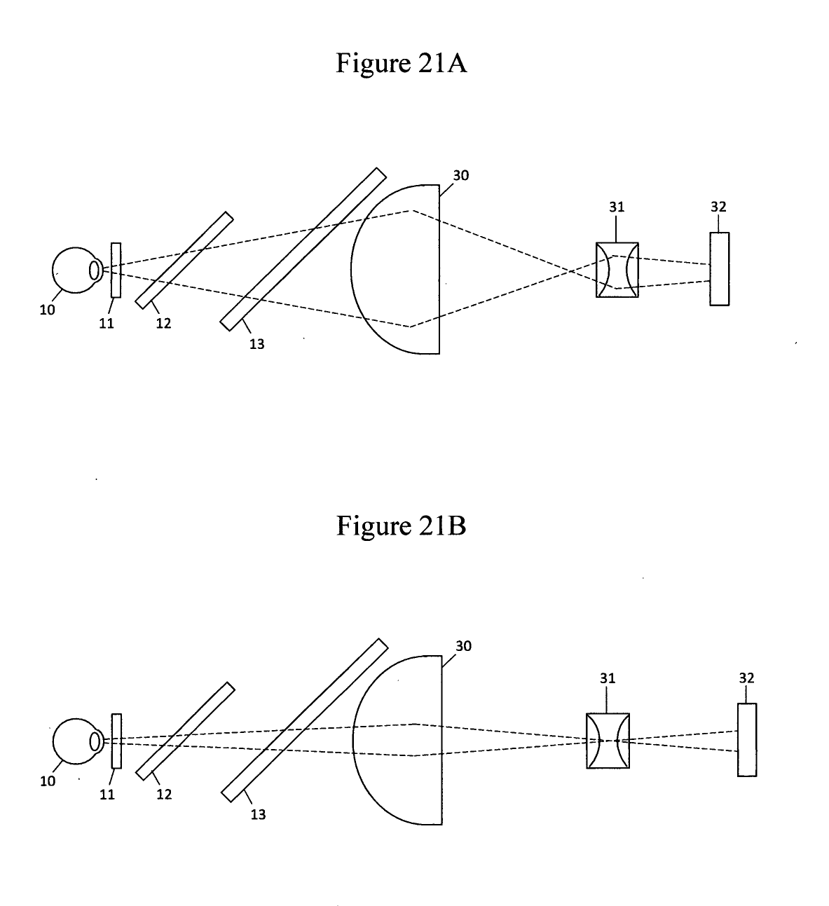

In a preferred embodiment, when viewing a microdisplay 32, the position of lens 30 and relay 31 with respect to lens 30 vary the apparent size of the display. Refer to FIG. 21A and FIG. 21B.

The present invention is well adapted to carry out the objective and attain the ends and advantages mentioned, as well as other ends and advantages inherent herein. While presently preferred embodiments of the invention have been given for the purpose of disclosure, numerous changes in the details of construction and arrangement of parts may be made without departing from the spirit of the invention.

1. A transparent device for imaging the eye.
a. Providing a viewport through the body of the structure of the camera.
b. Providing a hot mirror in the visual path tilted at 45 degrees.
c. Providing infrared illumination for the outside of the eye.
d. Providing infrared illumination for the inside of the eye.
e. Providing partially-reflective beamsplitter in the visual path tilted at 45 degrees.
f. Providing a visible light source to stimulate or bleach a portion of the viewer's retina.

2. The device of claim 1 wherein either or both eyes may be imaged

3. The device of claim 1 wherein the angle of the mirror and beamsplitter are other than 45 degrees.

4. The device of claim 1 wherein the light source is an LED.

5. The device of claim 1 wherein the light source is a laser.

6. The device of claim 1 wherein the camera captures an image suitable for commercial gaze-tracking algorithms.

7. The device of claim 1 wherein the camera captures a choroid image suitable for micro-tracking algorithms.

8. The device of claim 1 wherein the camera images are transferred by wired connection.

9. The system of claim 1 wherein the camera images are transferred by wireless transfer.

10. The device of claim 1 wherein camera imaging path aperture sizes are adjustable.

11. The device of claim 1 wherein the choroid camera and associated choroid illumination can be moved to view a different part of the eye without disturbing the viewer.

12. The system of claim 11 wherein the movement is caused by a remote controlled mechanism.

13. The device of claim 1 wherein the optical path is folded compact with mirrors.

14. The device of claim 1 wherein the front and back surfaces are constructed from infrared-transparent material for monitoring the subject.

15. The device of claim 1 wherein hardware is provided to constrain the motion of the viewer's head.

16. The device of claim 1 wherein a feedback device is provided.

17. The device of claim 1 wherein a microdisplay is provided for viewing

18. The system of claim 17 wherein the magnification of the microdisplay is variable

19. The system of claim 17 wherein the position of the microdisplay is variable

20. The device of claim 1 wherein a neutral density filter holder is provided in the viewer's path but not in the camera paths.
